(19) United States
(12) Patent Application Publication
(10) Pub. No.: US 2020/0221045 A1
(43) Pub. Date: Jul. 9, 2020

(54) IMAGING DEVICE AND ENDOSCOPE DEVICE

(71) Applicant: OLYMPUS CORPORATION, Tokyo (JP)

(72) Inventor: Yoshio Hagihara, Tokyo (JP)

(73) Assignee: OLYMPUS CORPORATION, Tokyo (JP)

(21) Appl. No.: 16/823,968

(22) Filed: Mar. 19, 2020

Related U.S. Application Data
(63) Continuation of application No. PCT/JP2017/036451, filed on Oct. 6, 2017.

(51) Int. Cl. H04N 5/3745; A61B 1/04; A61B 1/05; H04N 5/378; H04N 5/369

(52) U.S. Cl. CPC H04N 5/3745; A61B 1/042; H04N 5/379; H04N 5/378; A61B 1/051

(57) ABSTRACT

An imaging device includes a first substrate and a second substrate stacked on the first substrate. The first substrate includes a plurality of pixels. Each pixel block includes all of the pixels disposed in one or more columns in an array of the plurality of pixels. The second substrate includes a plurality of AD conversion circuits configured to convert a pixel signal read from two or more pixels belonging to the pixel block corresponding to the AD conversion circuit to a digital signal. For all combinations of two pixel blocks adjacent to each other in the first substrate, two AD conversion circuits corresponding to the adjacent two pixel blocks are adjacent to each other in the second substrate.

The present application is a continuation application based on International Patent Application No. PCT/JP2017/036451 filed on Oct. 6, 2017, the content of which is incorporated herein by reference.

### BACKGROUND OF THE INVENTION

### Field of the Invention

The present invention relates to an imaging device and an endoscope device.

### Description of Related Art

An imaging device is used for an imaging system such as a camera and a video device. Increasing pixels and speeding up an imaging device have been proceeding rapidly in recent years. The similar trend is seen in an imaging device used for an endoscope system.

A Variety of imaging devices such as a metal-oxide-semiconductor (MOS) type and a charge-coupled-device (CCD) type have been proposed and put into practical use so far. In addition, there is a so-called (C)MOS type imaging device including pixels constituting an amplification type solid-state imaging device (active pixel sensor (APS)) that amplifies and outputs a pixel signal in accordance with signal charge generated in a charge generation unit.

As an example of an imaging device of related art, a configuration disclosed in Japanese Unexamined Patent Application. First Publication No. 2005-347931 is known. Hereinafter, a configuration and an operation of the imaging device disclosed in Japanese Unexamined Patent Application, First Publication No. 2005-347931 will be described.

FIG. 10 shows a configuration of an imaging device 1000 disclosed in Japanese Unexamined Patent Application, First Publication No. 2005-347931. The imaging device 1000 includes an ADC 1106 including a comparator 1107 and a digital memory 1108 for each column of a pixel 1101. The ADC 1106 is an analog-to-digital (AD) conversion circuit. A digital value, i.e., a binary value output from a binary counter 1104 is input to a DAC 1105. The DAC 1105 is a digital-to-analog (DA) conversion circuit. The DAC 1105 generates a ramp voltage 1122 (ramp wave) in accordance with a digital value input thereto and outputs the ramp voltage 1122 to a first input unit of the comparator 1107. The ramp voltage is used as a reference signal.

A count value output from the binary counter 1104 is converted to a gray code by a binary converter 1115. A count value 1124 output from the binary converter 1115 is distributed to a digital memory 1108 of each column. A pixel signal read from the pixel 1101 to a signal line 1103 is input to a second input unit of the comparator 1107 in each ADC 1106 as an analog signal subject to AD conversion. A digital value held in the digital memory 1108 is converted to a binary value by a gray code converter 1116. A binary value 1126 output from the gray code converter 1116 is output to the outside of the imaging device 1000 through an output buffer 1109.

An AD conversion operation of the imaging device 1000 will be described. The binary counter 1104 starts counting in synchronization with a clock signal 1121 input from a clock generation circuit 1120. At the same time, the DAC 1105 starts generation of the ramp voltage 1122. The ramp voltage 1122 changes in synchronization with a count value of the binary counter 1104. A pixel signal read from the pixel 1101 of each column and the ramp voltage 1122 common between the columns are input to the comparator 1107 of each column. In parallel with this, the count value 1124 is distributed to the digital memory 1108.

When the magnitude relationship between two signals input to the comparator 1107 of a certain column has changed, an output voltage 1123 of the comparator 1107 is inverted and the digital memory 1108 of the column holds the count value 1124. The ramp voltage 1122 input to the comparator 1107 and the count value 1124 input to the digital memory 1108 are synchronized with each other. Therefore, through the above-described operation, AD conversion is performed on the pixel signal read from the pixel 1101 and a digital value is held in the digital memory 1108.

The above-described AD conversion method is a type particularly called ramp-type AD conversion (ramp run-up ADC). According to general classification of AD conversion methods, the above-described AD conversion method is a type called counting ADC (counting-type AD conversion). Using a ramp voltage (ramp wave) as a reference signal is equivalent to converting potential of an analog signal output from a pixel to length of time. AD conversion is realized by measuring length of time by using a clock signal having a fixed frequency.

Here, as an example of a specific device, an imager used in a camcorder or the like will be examined. Specifically, a specification is assumed that the number of pixels is 32,000,000 and the frame rate is 240 frames/sec. In order to make a description simple, it is assumed that the vertical and horizontal array of 32,000,000 pixels is constituted by 4000 rows and 8000 columns. For further simplification, it is assumed that no blanking period is present. Under these conditions, a reading rate of one row is represented by following Expression (1).

240 frames/sec×4000 rows/frame=960K rows/sec (1)

In other words, a reading rate of one row is 960 KHz. In a case in which the ADC included in the imaging device 1000 shown in FIG. 10 is applied to this device, the ADC needs to perform comparison 2¹² times, i.e., 4096 times in a reading period of one row for AD conversion of 12 bits. In other words, the ADC needs to change a count value output to the digital memory at a frequency of approximately 4 GHz that is 4000 times as large as the reading rate of one row. These conditions are unrealistic.

In this calculation, a waiting period until the ADC receives data from a pixel is not considered. In addition, a period for transferring an AD conversion result to an output memory, that is, a period in which the ADC is unable to perform a comparison operation is not considered. Moreover, since an optical black (OB) pixel period and a blanking period other than the above are excluded, a comparison operation at a higher frequency than that as estimated above is actually necessary.

In order to avoid the above-described problem, providing a plurality of ADCs for each column will be examined. For example, providing four ADCs for each column will be examined. In this way, a frequency of AD conversion of 12 bits is reduced to approximately 1 GHz and a frequency of AD conversion of 14 bits is reduced to approximately 4 GHz.

In the counting-type AD conversion method, it is possible to constitute an ADC by using a simple circuit. In addition, it is possible to lay out an ADC at a pixel pitch of approximately several micrometers by using current semiconductor process. However, in the counting-type AD conversion method, even when four ADCs are provided for each column, a clock of GHz order is necessary for a counting operation. Moreover, necessitating a comparator makes it hard to reduce noise.

In order to solve the above-described problem, an ADC disclosed in a non-patent document (A. Matsuzawa and M. Miyahara, “A SAR-AE ADC with Dynamic Integrator for Low-Noise CMOS Image Sensors,” Proc. 2017 IISW, pp. 324-327) may be used instead of the counting-type AD conversion method. In the non-patent document, a configuration using a combination of a successive approximation register ADC and a ΔΣ ADC is disclosed. This configuration makes it possible to reduce noise.

In order to achieve a further increase in the number of pixels of an imaging device, an imaging device including plurality of laminated substrates has been developed. In this imaging device, a plurality of pixels are disposed in a first substrate and an ADC is disposed in a second substrate. Due to this configuration, it is possible to expand an area of a pixel region.

### SUMMARY OF THE INVENTION

According to a first aspect of the present invention, an imaging device includes a first substrate and a second substrate stacked on the first substrate. The first substrate includes a plurality of pixels forming a first array and disposed in a matrix shape. Each pixel included in the plurality of pixels belongs to one pixel block included in a plurality of pixel blocks and is configured to output an analog pixel signal. The second substrate includes a plurality of AD conversion circuits forming a second array. Each AD conversion circuit included in the plurality of AD conversion circuits is configured to convert the pixel signal read from two or more pixels included in the plurality of pixels and belonging to the pixel block corresponding to the AD conversion circuit to a digital signal. At least one of the first substrate and the second substrate includes a selecting circuit configured to control a timing at which the pixel signal is read from the plurality of pixels. Each pixel block included in the plurality of pixel blocks includes all of the pixels disposed in one or more columns in the first array. The plurality of AD conversion circuits are disposed in a matrix shape having M rows and N columns. The number M is an integer greater than or equal to 3. The number N is an integer greater than or equal to 2. A width of each AD conversion circuit included in the plurality of AD conversion circuits in a row direction in the second array is larger than a pitch of each pixel included in the plurality of pixels. For all combinations of two pixel blocks included in the plurality of pixel blocks and adjacent to each other in the first substrate, two AD conversion circuits included in the plurality of AD conversion circuits and corresponding to the adjacent two pixel blocks are adjacent to each other in the second substrate.

According to a second aspect of the present invention, in the first aspect, the plurality of pixel blocks may include a first pixel block, a second pixel block, a third pixel block, and a fourth pixel block. The second pixel block may be adjacent to the first pixel block in a row direction in the first array. The fourth pixel block may be adjacent to the third pixel block in the row direction in the first array. The plurality of AD conversion circuits may include a first AD conversion circuit corresponding to the first pixel block, a second AD conversion circuit corresponding to the second pixel block, a third AD conversion circuit corresponding to the third pixel block, and a fourth AD conversion circuit corresponding to the fourth pixel block. The second AD conversion circuit may be adjacent to the first AD conversion circuit in a column direction in the second array. The fourth AD conversion circuit may be adjacent to the third AD conversion circuit in a direction opposite to the column direction in the second array. A first column including the first AD conversion circuit and the second AD conversion circuit may be adjacent to a second column including the third AD conversion circuit and the fourth AD conversion circuit in the second array.

According to a third aspect of the present invention, in the first or second aspect, the imaging device may further include a connection electrode electrically connecting the first substrate and the second substrate together. The pixel that belongs to each pixel block included in the plurality of pixel blocks may be connected to a signal line disposed in the first substrate. The connection electrode may be disposed so as to overlap the AD conversion circuit included in the plurality of AD conversion circuits and may be connected to the signal line. Each AD conversion circuit included in the plurality of AD conversion circuits may be connected to the connection electrode.

According to a fourth aspect of the present invention, an imaging device includes a first substrate and a second substrate stacked on the first substrate. The first substrate includes a plurality of pixels forming a first array and disposed in a matrix shape. Each pixel included in the plurality of pixels belongs to one pixel block included in a plurality of pixel blocks and is configured to output an analog pixel signal. The second substrate includes a plurality of AD conversion circuits forming a second array.

Each AD conversion circuit included in the plurality of AD conversion circuits is configured to convert the pixel signal read from two or more pixels included in the plurality of pixels and belonging to the pixel block corresponding to the AD conversion circuit to a digital signal. At least one of the first substrate and the second substrate includes a selecting circuit configured to control a timing at which the pixel signal is read from the plurality of pixels. Each pixel block included in the plurality of pixel blocks includes all of the pixels disposed in one or more columns in the first array. The plurality of AD conversion circuits are disposed in a matrix shape having M rows and N columns. The number M is an integer greater than or equal to 3. The number N is an integer greater than or equal to 2. A width of each AD conversion circuit included in the plurality of AD conversion circuits in a row direction in the second array is larger than a pitch of each pixel included in the plurality of pixels. For all combinations of two AD conversion circuits included in the plurality of AD conversion circuits and adjacent to each other in a column direction in the second array, two pixel blocks included in the plurality of pixel blocks and corresponding to the two AD conversion circuits adjacent to each other in the column direction are adjacent to each other in the first substrate. For all the combinations, the two AD conversion circuits adjacent to each other in the column direction are shifted from each other by a predetermined distance in a row direction in the second array.

According to a fifth aspect of the present invention, in the fourth aspect, the predetermined distance may be integer times as large as the pitch.

According to a sixth aspect of the present invention, in the fourth or fifth aspect, the imaging device may further include a connection electrode electrically connecting the first substrate and the second substrate together. Shapes of any two AD conversion circuits included in the plurality of AD conversion circuits may be the same. Areas of any two AD conversion circuits included in the plurality of AD conversion circuits may be the same. Each AD conversion circuit included in the plurality of AD conversion circuits may be connected to the pixel block corresponding to the AD conversion circuit through the connection electrode. Each AD conversion circuit included in the plurality of AD conversion circuits may be connected to the connection electrode at the same position in the AD conversion circuit.

According to a seventh aspect of the present invention, in any one of the first to sixth aspects, each AD conversion circuit included in the plurality of AD conversion circuits may be configured to be an AD conversion circuit using a ΔΣ method.

According to an eighth aspect of the present invention, an endoscope device includes the imaging device.

### BRIEF DESCRIPTION OF THE DRAWINGS

FIG. 1 is a diagram showing a configuration of an imaging device according to a first embodiment of the present invention.

FIG. 2 is a block diagram showing a circuit configuration of a first substrate and a second substrate in the imaging device according to the first embodiment of the present invention.

FIG. 3 is a circuit diagram showing a configuration of a pixel in the imaging device according to the first embodiment of the present invention.

FIG. 4 is a block diagram showing a circuit configuration of a first substrate and a second substrate in an imaging device according to a second embodiment of the present invention.

FIG. 5 is a block diagram showing a circuit configuration of a first substrate and a second substrate in an imaging device according to a third embodiment of the present invention.

FIG. 6 is a block diagram showing arrangement of an AD conversion circuit in an embodiment compared to the third embodiment of the present invention.

FIG. 7 is a block diagram showing a circuit configuration of a first substrate and a second substrate in an imaging device according to a fourth embodiment of the present invention.

FIG. 8 is a circuit diagram showing a configuration of a pixel in the imaging device according to the fourth embodiment of the present invention.

FIG. 9 is a block diagram showing a configuration of an endoscope device according to a fifth embodiment of the present invention.

FIG. 10 is a block diagram showing a configuration of an imaging device of the related art.

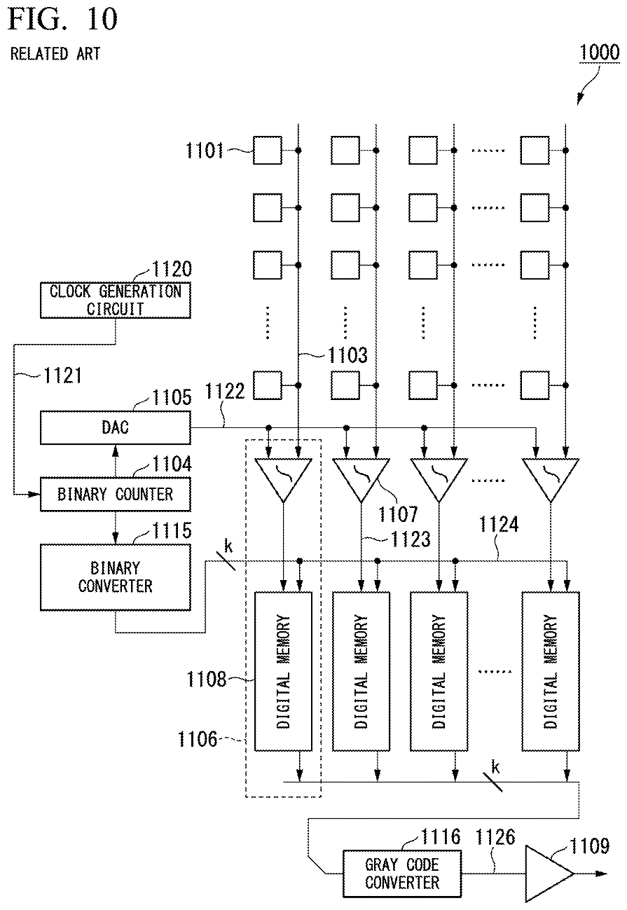

### DETAILED DESCRIPTION OF THE INVENTION

Hereinafter, embodiments of the present invention will be described with reference to the drawings.

### First Embodiment

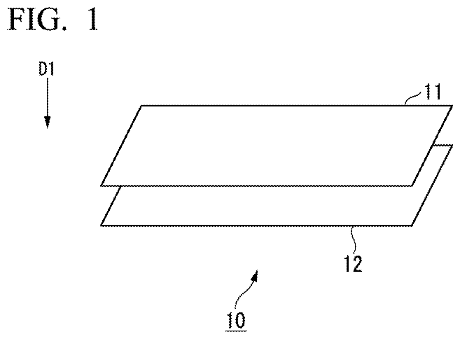

FIG. 1 shows a configuration of an imaging device 10 according to a first embodiment of the present invention. As shown in FIG. 1, the imaging device 10 includes a first substrate 11 and a second substrate 12 stacked on the first substrate 11. The second substrate 12 is stacked in a stacking direction D1 with respect to the first substrate 11. The stacking direction D1 is a direction perpendicular to a main surface of the first substrate 11 or the second substrate 12. For example, the first substrate 11 and the second substrate 12 are connected with each other through Cu—Cu bonding or the like.

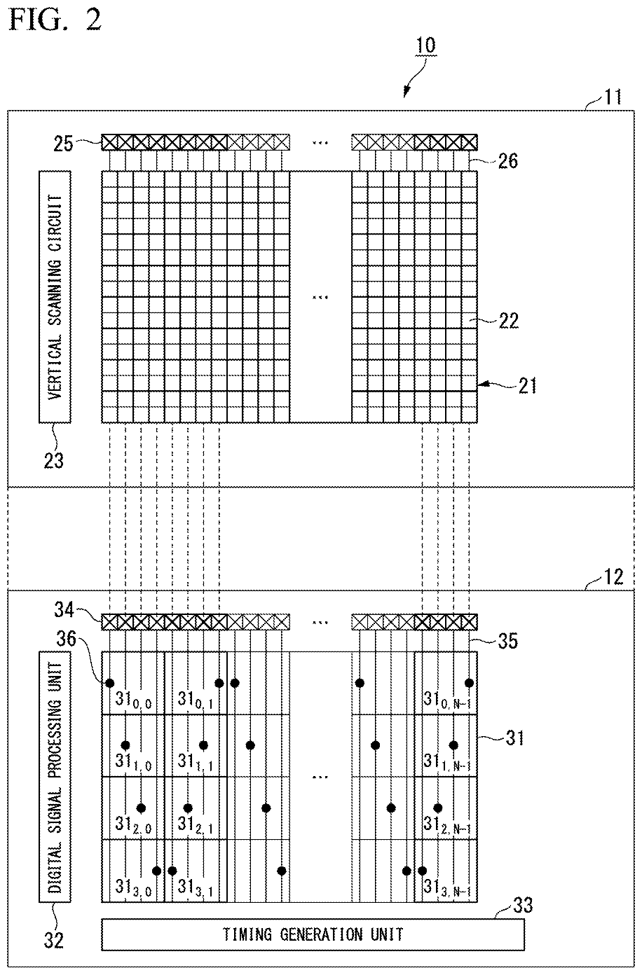

FIG. 2 shows a circuit configuration of the first substrate 11 and the second substrate 12. In FIG. 2, planar arrangement of circuits in the first substrate 11 and the second substrate 12 is shown. As shown in FIG. 2, the first substrate 11 includes an imaging unit 21 and a vertical scanning circuit 23.

The imaging unit 21 includes a plurality of pixels 22 disposed in a matrix shape. Each pixel 22 included in the plurality of pixels 22 belongs to any one of a plurality of pixel blocks and outputs an analog pixel signal. For example, the pixel 22 has a rectangular shape. Shapes and areas of the plurality of pixels 22 are the same between the pixels 22.

A row direction in the array of the plurality of pixels 22 is a lateral direction (horizontal direction) in FIG. 2. A column direction in the array of the plurality of pixels 22 is different from the row direction. The column direction in the array of the plurality of pixels 22 is a longitudinal direction (vertical direction) in FIG. 2. The number of pixels in the row direction is m. The number m is an integer greater than or equal to 6. The number of pixels in the column direction is greater than or equal to 2. In the example shown in FIG. 2, the number of pixels in the column direction is 16.

Each pixel block included in the plurality of pixel blocks includes all of the pixels 22 disposed in one or more columns in the array of the plurality of pixels 22. In the example shown in FIG. 2, each pixel block includes the pixels 22 of one column. All of the pixels 22 disposed in the same column are included in the same pixel block. For example, the pixels 22 in the most left column in FIG. 2 belongs to a first pixel block. The pixels 22 in the k-th column from the left belongs to a k-th pixel block. The number k is any integer greater than or equal to 1 and less than or equal to m. In the example shown in FIG. 2, the number of columns of the plurality of pixels 22 and the number of pixel blocks are the same.

A plurality of vertical signal lines 26 are disposed in the first substrate 11. Each vertical signal line 26 corresponds to each column in the array of the plurality of pixels 22. In the example shown in FIG. 2, the number of columns of the plurality of pixels 22 and the number of vertical signal lines 26 are the same. The vertical signal lines 26 are disposed to extend in the column direction. One vertical signal line 26 is connected to all of the pixels 22 disposed in one column. All of the pixels 22 disposed in the same column are connected to the same vertical signal line 26.

A plurality of connection electrodes 25 are disposed in the first substrate 11. Each connection electrode 25 corresponds to each vertical signal line 26. In the example shown in FIG. 2, the number of vertical signal lines 26 and the number of connection electrodes 25 are the same. For example, the connection electrode 25 constitutes Cu—Cu bonding. The connection electrode 25 may include a bump. The connection electrode 25 may include a wiring and a via.

The connection electrodes 25 are disposed outside the imaging unit 21. In the example shown in FIG. 2, the connection electrodes 25 are disposed to the upper side of the imaging unit 21. The connection electrodes 25 may be disposed to the lower, right, or left side of the imaging unit 21. The connection electrodes 25 may be disposed inside the imaging unit 21.

The pixel 22 outputs a pixel signal to the vertical signal line 26. The pixel signal output from the pixel 22 is transferred to the connection electrode 25 by the vertical signal line 26. The connection electrode 25 transfers the pixel signal to the second substrate 12.

The vertical scanning circuit 23 is disposed outside the imaging unit 21. In the example shown in FIG. 2, the vertical scanning circuit 23 is disposed to the left side of the imaging unit 21. The vertical scanning circuit 23 may be disposed to the right side of the imaging unit 21. The vertical scanning circuit 23 is long in the column direction. The vertical scanning circuit 23 controls a timing at which a pixel signal is read from the plurality of pixels 22. The vertical scanning circuit 23 controls an operation of the plurality of pixels 22 by outputting a control signal to the plurality of pixels 22. The vertical scanning circuit 23 outputs a control signal for each row in the array of the plurality of pixels 22.

The vertical scanning circuit 23 may be disposed in the second substrate 12. The vertical scanning circuit 23 may include a first vertical scanning circuit disposed in the first substrate 11 and a second vertical scanning circuit disposed in the second substrate 12. The imaging device 10 may include a horizontal scanning circuit that controls reading of a pixel signal for each column. The horizontal scanning circuit is disposed in the first substrate 11 or the second substrate 12. The horizontal scanning circuit may include a first horizontal scanning circuit disposed in the first substrate 11 and a second horizontal scanning circuit disposed in the second substrate 12. Therefore, at least one of the first substrate 11 and the second substrate 12 has only to include a scanning circuit.

As shown in FIG. 2, the second substrate 12 includes a plurality of ADCs 31, a digital signal processing unit 32, and a timing generation unit 33.

The plurality of ADCs 31 are disposed in a matrix shape. Specifically, the plurality of ADCs 31 are disposed in a matrix shape having M rows and N columns. The number M is an integer greater than or equal to 3 and the number N is an integer greater than or equal to 2. In the example shown in FIG. 2, the number of rows of the plurality of ADCs 31 is 4. The number of columns of the plurality of ADCs 31 is m/4. For example, the ADC 31 has a rectangular shape. Shapes and areas of the plurality of ADCs 31 are the same between the ADCs 31. The plurality of ADCs 31 convert pixel signals read from two or more pixels 22 belonging to the corresponding pixel block to digital signals.

The row direction in the array of the plurality of ADCs 31 is the same as the row direction in the array of the plurality of pixels 22. The column direction in the array of the plurality of ADCs 31 is different from the row direction. The column direction in the array of the plurality of ADCs 31 is the same as the column direction in the array of the plurality of pixels 22.

The plurality of ADCs 31 are disposed in a region corresponding to a pixel region in which the plurality of pixels 22 are disposed. When the first substrate 11 and the second substrate 12 are seen in the stacking direction D1, at least part of the pixel region and at least part of the region in which the plurality of ADCs 31 are disposed overlap each other.

The width of each ADC 31 included in the plurality of ADCs 31 in the row direction in the array of the plurality of ADCs 31 is larger than the pitch of the pixel 22. The pitch of the pixel 22 is the width of the pixel 22 in the row direction. For example, the width of the ADC 31 in the row direction is two or more times as large as the pitch of the pixel 22. In the example shown in FIG. 2, the width of the ADC 31 in the row direction is four times as large as the pitch of the pixel 22. Therefore, the width of the ADC 31 in the row direction is equal to the sum of the widths of four pixels 22 in the row direction.

In FIG. 2, the ADC 31 disposed in the p-th row from the top and the q-th column from the left is represented as “ADC 31_{p-1,q-1}.” For example, the upper left ADC 31 in the array of the plurality of ADCs 31 is an ADC 31_{0,0}. The upper right ADC 31 in the array of the plurality of ADCs 31 is an ADC 31_{0,N-1}. The number N is an integer greater than or equal to 2 and less than or equal to m/4. The lower left ADC 31 in the array of the plurality of ADCs 31 is an ADC 31_{3,0}. The lower right ADC 31 in the array of the plurality of ADCs 31 is an ADC 31_{3,N-1}.

For all the combinations of two pixel blocks adjacent to each other in the first substrate 11, two ADCs 31 corresponding to the two pixel blocks adjacent to each other are adjacent to each other in the second substrate 12. Two ADCs 31 corresponding to the two pixel blocks adjacent to each other in the row direction are adjacent to each other in the column direction or the row direction.

Hereinafter, an example in which codes are given to the pixel blocks will be described in order to distinguish the pixel blocks. The plurality of pixel blocks include four pixel blocks consisting of a pixel block A (first pixel block), a pixel block B (second pixel block), a pixel block C, and a pixel block D corresponding to four consecutive columns. The pixel block B is adjacent to the pixel block A in the row direction in the array of the plurality of pixels 22. In the example shown in FIG. 2, the row direction is the right direction. The pixel block C is adjacent to the pixel block B in the row direction. The pixel block D is adjacent to the pixel block C in the row direction. The four pixel blocks correspond to the pixels 22 of the first to fourth columns from the left.

The plurality of ADCs 31 include an ADC 31_{0,0} corresponding to the pixel block A, an ADC 31_{1,0} corresponding to the pixel block B, an ADC 31_{2,0} corresponding to the pixel block C (first AD conversion circuit), and an ADC 31_{1,0} corresponding to the pixel block D (second AD conversion circuit). The ADC 31_{1,0} is adjacent to the ADC 31_{0,0} in the column direction in the array of the plurality of ADCs 31. In the example shown in FIG. 2, the column direction is the lower direction. The ADC 31_{2,0} is adjacent to the ADC 31_{1,0} in the column direction. The ADC 31_{3,0} is adjacent to the ADC 31_{2,0} in the column direction.

The plurality of pixel blocks include four pixel blocks consisting of a pixel block E (third pixel block), a pixel block F (fourth pixel block), a pixel block G and a pixel block H corresponding to four consecutive columns. The pixel block E is adjacent to the pixel block D in the row direction in the array of the plurality of pixels 22. The pixel block F is adjacent to the pixel block E in the row direction. The pixel block G is adjacent to the pixel block F in the row direction. The pixel block H is adjacent to the pixel block G in the row direction. The four pixel blocks correspond to the pixels 22 of the fifth to eighth columns from the left.

The plurality of ADCs 31 include an ADC 31_{3,1} (third AD conversion circuit) corresponding to the pixel block E, an ADC 31_{2,1} (fourth AD conversion circuit) corresponding to the pixel block F, an ADC 31_{1,1} corresponding to the pixel block (and an ADC 31_{0,1} corresponding to the pixel block H. The ADC 31_{3,1} is adjacent to the ADC 31_{3,0} in the row direction in the array of the plurality of ADCs 31. The ADC 31_{2,1} is adjacent to the ADC 31_{3,1} in the direction opposite to the column direction in the array of the plurality of ADCs 31. In the example shown in FIG. 2, the direction opposite to the column direction is the upper direction. The ADC 31_{1,1} is adjacent to the ADC 31_{2,1} in the direction opposite to the column direction. The ADC 31_{0,1} is adjacent to the ADC 31_{1,1} in the direction opposite to the column direction.

In the array of the plurality of ADCs 31, a first column includes the ADC 31_{0,0}, the ADC 31_{1,0}, the ADC 31_{2,0}, and the ADC 31_{3,0}. In the array of the plurality of ADCs 31, a second column includes the ADC 31_{0,1}, the ADC 31_{1,1} the ADC 31_{2,1}, and the ADC 31_{3,1}. The first column is adjacent to the second column.

As the column in the four pixel blocks corresponding to the first column proceeds, the row in four ADCs 31 constituting the first column proceeds in a first direction. In the example shown in FIG. 2, the direction in which the column in the four pixel blocks proceeds is the right direction. In the example shown in FIG. 2, the first direction is the lower direction. As the column in the four pixel blocks corresponding to the second column proceeds, the row in four ADCs 31 constituting the second column proceeds in a second direction opposite to the first direction. In the example shown in FIG. 2, the second direction is the upper direction.

The first column and the second column are alternately disposed in the row direction in the array of the plurality of ADCs 31. In other words, the first column and the second column are periodically disposed. In the first column disposed in the right side of the second column, an ADC 31_{0,2} is adjacent to the ADC 31_{0,1} in the row direction in the array of the plurality of ADCs 31.

A pseudo (dummy) ADC may be disposed. For example, a plurality of pseudo (dummy) ADCs may be disposed to surround the plurality of ADCs 31.

A plurality of vertical signal lines 35 are disposed in the second substrate 12. Each vertical signal line 35 corresponds to each column in the array of the plurality of pixels 22. In the example shown in FIG. 2, the number of columns of the plurality of pixels 22 and the number of vertical signal lines 35 are the same. The vertical signal lines 35 are disposed to extend in the column direction. The vertical signal lines 35 are disposed at positions corresponding to the vertical signal lines 26 in the second substrate 12. One vertical signal line 35 is connected to one of the ADCs 31 disposed in one column. The ADC 31 includes an input terminal 36 to which a pixel signal output from the pixel 22 of the corresponding pixel block is input. The input terminal 36 is connected to the vertical signal line 35.

In this example, the position at which each input terminal 36 in the four ADCs 31 constituting the first column is disposed and the position at which each input terminal 36 in the four ADCs 31 constituting the second column is disposed are line-symmetric with each other. Therefore, the layout of the four ADCs 31 constituting the first column and the layout of the four ADCs 31 constituting the second column are line-symmetric with each other.

The ADC 31_{0,1}, is constituted similarly to the ADC 31_{3,0}. The ADC 31_{1,1} is constituted similarly to the ADC 31_{2,0}. The ADC 31_{2,1} is constituted similarly to the ADC 31_{1,0}. The ADC 31_{3,1} is constituted similarly to the ADC 31_{0,0}. Therefore, each column in the array of the plurality of ADCs 31 is a combination of the ADCs 31 of four patterns.

A plurality of connection electrodes 34 are disposed in the second substrate 12. Each connection electrode 34 corresponds to each vertical signal line 35. In the example shown in FIG. 2, the number of vertical signal lines 35 and the number of connection electrodes 34 are the same. The connection electrodes 34 are disposed at positions corresponding to the connection electrodes 25 in the second substrate 12. When the first substrate 11 and the second substrate 12 are seen in the stacking direction D1, at least part of the connection electrode 25 and at least part of the connection electrode 34 overlap each other. The connection electrode 34 is electrically connected to the connection electrode 25. For example, the connection electrode 34 constitutes Cu—Cu bonding. The connection electrode 34 may include a bump. The connection electrode 34 may include a wiring and a via. Although the connection electrode 25 and the connection electrode 34 are independent of each other in the example shown in FIG. 2, the connection electrode 25 and the connection electrode 34 may be integrated together. The connection electrode 25 and the connection electrode 34 electrically connect the first substrate 11 and the second substrate 12 together.

The connection electrodes 34 are disposed outside the plurality of ADCs 31. In the example shown in FIG. 2, the connection electrodes 34 are disposed to the upper side of the plurality of ADCs 31. The connection electrodes 34 may be disposed to the lower, right, or left side of the plurality of ADCs 31. The connection electrodes 34 may be disposed in the region of the plurality of ADCs 31.

A pixel signal output from the pixel 22 is transferred to the second substrate 12 by the connection electrode 25 and the connection electrode 34. A pixel signal output from the connection electrode 34 is transferred to the ADC 31 by the vertical signal line 35.

The digital signal processing unit 32 processes a digital signal generated by the ADC 31. The timing generation unit 33 generates a timing signal for controlling the vertical scanning circuit 23, the ADC 31, and the digital signal processing unit 32.

The positions at which the digital signal processing unit 32 and the timing generation unit 33 are disposed in the second substrate 12 are not limited to the positions shown in FIG. 2. The digital signal processing unit 32 and the timing generation unit 33 may be disposed in the first substrate 11. The digital signal processing unit 32 may include a first digital signal processing unit disposed in the first substrate 11 and a second digital signal processing unit disposed in the second substrate 12. The timing generation unit 33 may include a first timing generation unit disposed in the first substrate 11 and a second timing generation unit disposed in the second substrate 12. The digital signal processing unit 32 and the timing generation unit 33 may be a circuit disposed outside the imaging device 10.

The first substrate 11 and the second substrate 12 are connected together in the periphery of each substrate. For example, in a case in which a third substrate including a memory is stacked between the first substrate 11 and the second substrate 12, connection between the first substrate 11 and the third substrate or connection between the second substrate 12 and the third substrate becomes simplified.

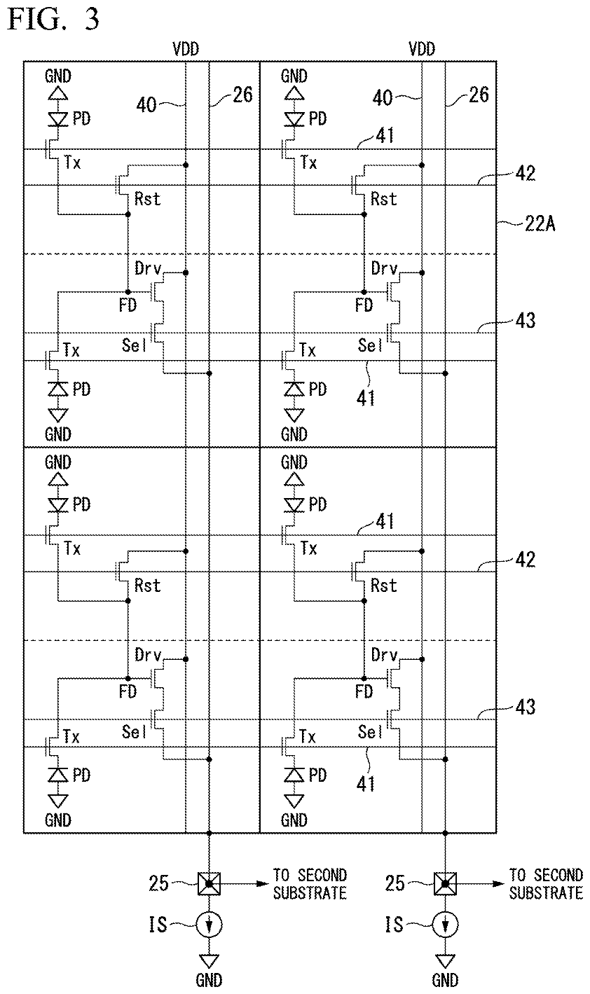

FIG. 3 shows a configuration of the pixel 22. Configurations of the eight pixels 22 are shown in FIG. 3 as a representative. Configurations of other pixels 22 are similar to the configuration shown in FIG. 3. Two pixels 22 adjacent to each other in the column direction constitute a shared pixel 22A. The shared pixel 22A shares part of the circuits included in each pixel 22.

The pixel 22 includes a photodiode PD, a transfer transistor Tx, a floating diffusion FD, a reset transistor Rst, an amplification transistor Dry, and a selection transistor Sel. Each transistor shown in FIG. 3 is an NMOS transistor. Each transistor shown in FIG. 3 includes a gate terminal, a source terminal, and a drain terminal.

The photodiode PD includes a first terminal and a second terminal. The first terminal of the photodiode PD is connected to a ground GND. The second ternminal of the photodiode PD is connected to the transfer transistor Tx.

The drain terminal of the transfer transistor Tx is connected to the second terminal of the photodiode PD. The source terminal of the transfer transistor Tx is connected to the floating diffusion FD. The gate terminal of the transfer transistor Tx is connected to a control signal line 41. The control signal line 41 is connected to the vertical scanning circuit 23. A transfer pulse output from the vertical scanning circuit 23 is input to the gate terminal of the transfer transistor Tx.

The drain terminal of the reset transistor Rst is connected to a power source line 40. The power source line 40 is connected to a power source that outputs a power source voltage VDD. The source terminal of the reset transistor Rst is connected to the floating diffusion FD. The gate terminal of the reset transistor Rst is connected to a control signal line 42. The control signal line 42 is connected to the vertical scanning circuit 23. A reset pulse output from the vertical scanning circuit 23 is input to the gate terminal of the reset transistor Rst.

The drain terminal of the amplification transistor Dry is connected to the power source line 40. The source terminal of the amplification transistor Dry is connected to the selection transistor Sel. The gate terminal of the amplification transistor Dry is connected to the floating diffusion FD.

The drain terminal of the selection transistor Set is connected to the source terminal of the amplification transistor Dry. The source terminal of the selection transistor Sel is connected to the vertical signal line 26. The gate terminal of the selection transistor Sel is connected to a control signal line 43. The control signal line 43 is connected to the vertical scanning circuit 23. A selection pulse output from the vertical scanning circuit 23 is input to the gate terminal of the selection transistor Sel.

The transfer transistor Tx is controlled by using the transfer pulse output from the vertical scanning circuit 23. The reset transistor Rst is controlled by using the reset pulse output from the vertical scanning circuit 23. The selection transistor Sel is controlled by using the selection pulse output from the vertical scanning circuit 23.

The photodiode FD generates signal charge in accordance with the amount of incident light. The transfer transistor Tx transfers the signal charge generated in the photodiode PD to the floating diffusion FD. The floating diffusion PD accumulates the signal charge transferred by the transfer transistor Tx. The reset transistor Rst resets the voltage of the floating diffusion FD to a predetermined voltage. The amplification transistor Dry generates a pixel signal by amplifying a signal in accordance with the voltage of the floating diffusion FD. The selection transistor Sel outputs the pixel signal to the vertical signal line 26. A current source IS is connected to the vertical signal line 26.

Two pixels 22 constituting the shared pixel 22A share the floating diffusion FD, the reset transistor Rst, the amplification transistor Dry, and the selection transistor Sel. The number of pixels 22 constituting the shared pixel 22A is not limited to two. Each pixel 22 may not share a circuit with another pixel 22. For example, the pixel 22 can be constituted by using the backside-irradiation-type (BSI) technology.

There is a case in which the variation of property between two ADCs 31 far apart from each other is large due to the variation of a production process. In the first embodiment, two ADCs 31 corresponding to two pixel blocks adjacent to each other are adjacent to each other. The variation of property is small between two ADCs 31 adjacent to each other. Therefore, the difference of influence due to the variation of property between two ADCs 31 is small between two pixel signals corresponding to two columns adjacent to each other. For this reason, the imaging device 10 can suppress deterioration of image quality due to the variation of property between the ADCs 31.

It is assumed that the vertical and horizontal array of the pixels 22 is constituted by 4000 rows and 8000 columns and the pitch of the pixels 22 is 2.5 μm. In a case in which the area of one ADC 31 corresponds to 500 pixels in the column direction and 8 pixels in the row direction, the width of the ADC 31 in the column direction is 1250 μm and the width of the ADC 31 in the row direction is 20 μm. In a case in which the AD conversion rate of a pixel signal is assumed to be 1 MHz, time required for AD conversion of a pixel signal corresponding to one pixel 22 is 1 μsec. In other words, time required for one ADC 31 to perform AD conversion for 500×8 pixels is 4 msec. Therefore, a frame rate exceeding 240 frames/sec can be realized in an imaging device of which the number of pixels is 32,000,000.

The plurality of ADCs 31 may be an AD conversion circuit using a ΔΣ method. In this way, the imaging device 10 can reduce noise to the degree of 20 μV. As a consequence, the imaging device 10 can realize AD conversion of 14 bits.

The layout of the four ADCs 31 constituting the first column and the layout of the four ADCs 31 constituting the second column are line-symmetric with each other. In this way, the number of patterns of the ADCs 31 constituting each column in the array of the plurality of ADCs 31 is reduced. In other words, laying out the ADCs 31 is easy.

### Second Embodiment

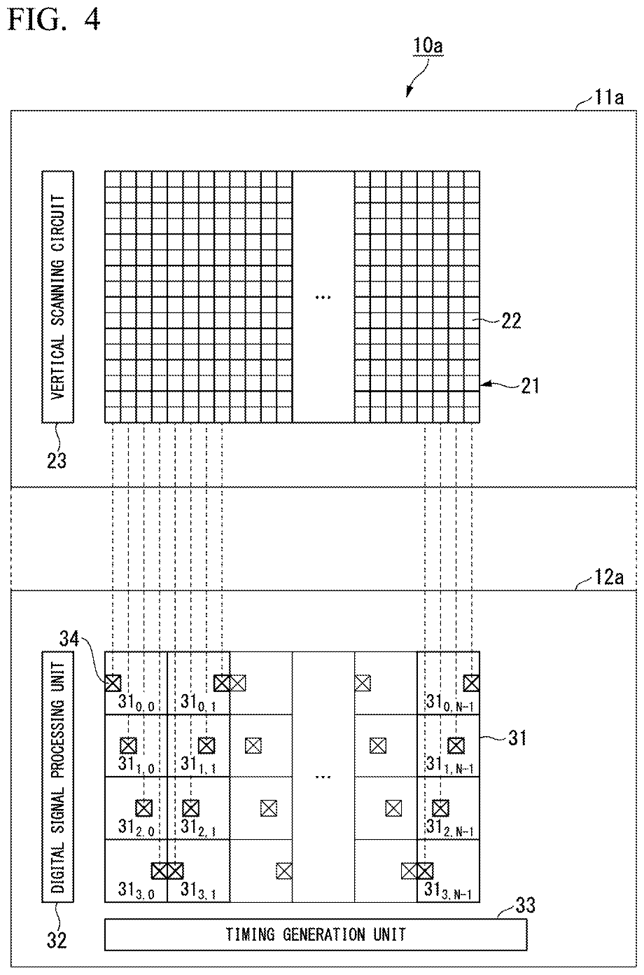

FIG. 4 shows a circuit configuration of a first substrate 11a and a second substrate 12a included in an imaging device 10a according to a second embodiment of the present invention. In FIG. 4, planar arrangement of circuits in the first substrate 11a and the second substrate 12a is shown. In terms of the configuration shown in FIG. 4, points different from the configuration shown in FIG. 2 will be described.

In the first substrate 11a, a connection electrode 25 is disposed in an imaging unit 21. For the convenience of drawing, the connection electrode 25 and a vertical signal line 26 are not shown in FIG. 4.

In the second substrate 12a, a connection electrode 34 is disposed in a region of a plurality of ADCs 31. For example, the connection electrode 34 is disposed at a position overlapping an input terminal 36 of the ADC 31. For the convenience of drawing, the input terminal 36 of the ADC 31 is not shown in FIG. 4. A vertical signal line 35 is not disposed. In the first substrate 11a, the connection electrode 25 is disposed at a position overlapping the connection electrode 34.

The connection electrode 25 and the connection electrode 34 electrically connect the first substrate 11a and the second substrate 12a together. Each pixel 22 that belongs to each pixel block included in a plurality of pixel blocks is connected to the vertical signal line 26 disposed in the first substrate 11a. The connection electrode 25 and the connection electrode 34 are disposed so as to overlap the ADC 31 and are connected to the vertical signal line 26. Each ADC 31 included in the plurality of ADCs 31 is connected to the connection electrode 34.

In terms of points other than the above, the configuration shown in FIG. 4 is similar to the configuration shown in FIG. 2.

The imaging device 10a according to the second embodiment can suppress deterioration of image quality due to the variation of property between the ADCs 31 as with the imaging device 10 according to the first embodiment. In the second embodiment, an effect similar to that in the first embodiment can be obtained.

The ADC 31 is connected to the vertical signal line 26 through the connection electrode 25 and the connection electrode 34 that are disposed so as to overlap the ADC 31. In this way, the vertical signal line 35 is unnecessary, thus shortening a signal line for transferring a pixel signal.

### Third Embodiment

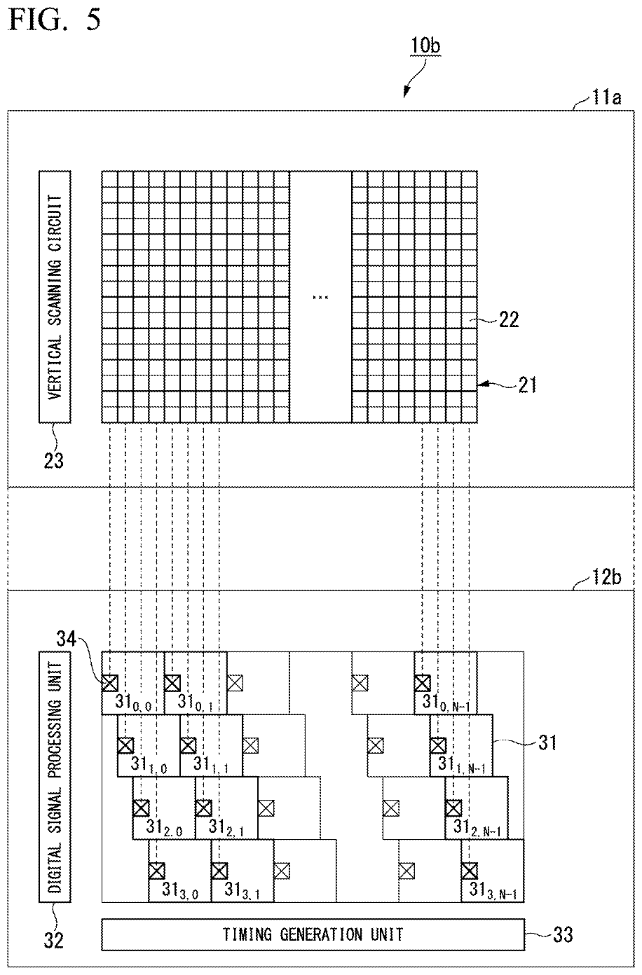

FIG. 5 shows a circuit configuration of a first substrate 11a and a second substrate 12b included in an imaging device 10b according to a third embodiment of the present invention. In FIG. 5, planar arrangement of circuits in the first substrate 11a and the second substrate 12b is shown. In terms of the configuration shown in FIG. 5, points different from the configuration shown in FIG. 4 will be described.

The first substrate 11a shown in FIG. 5 is the same as the first substrate 11a shown in FIG. 4. For all the combinations of two ADCs 31 adjacent to each other in a column direction in the array of the plurality of ADCs 31, two pixel blocks corresponding to two ADCs 31 adjacent to each other in the column direction are adjacent to each other in the first substrate 11a. For all the combinations of two ADCs 31 adjacent to each other in the column direction, two ADCs 31 adjacent to each other in the column direction are shifted from each other by a predetermined distance in a row direction in the array of the plurality of ADCs 31.

The column direction in the array of the plurality of ADCs 31 is tilted by a predetermined angle with respect to a direction (lower direction) perpendicular to the row direction. The predetermined angle is greater than 0 degree and less than 90 degrees.

The predetermined distance is integer times as large as the pitch of the pixel 22. For example, the integer is any one of 1 to 4. The predetermined distance is smaller than the width of the ADC 31 in the row direction in the array of the plurality of ADCs 31.

For example, two image blocks corresponding to an ADC 31_{0,0} and an ADC 31_{1,0} adjacent to each other in the column direction are adjacent to each other in the row direction in the first substrate 11a. The ADC 31_{1,0} is shifted from the ADC 31_{0,0} by a pixel pitch in the row direction. Two image blocks corresponding to the ADC 31_{1,0} and an ADC 31_{2,0} adjacent to each other in the column direction are adjacent to each other in the row direction in the first substrate 11a. The ADC 31_{2,0} is shifted from the ADC 31_{1,0} by the pixel pitch in the row direction. Two image blocks corresponding to the ADC 31_{2,0} and an ADC 31_{3,0} adjacent to each other in the column direction are adjacent to each other in the row direction in the first substrate 11a. The ADC 31_{3,0} is shifted from the ADC 31_{2,0} by the pixel pitch in the row direction. As described above, row direction positions of two ADCs 31 adjacent to each other in the column direction are different from each other by a predetermined distance.

The connection electrode 25 and the connection electrode 34 electrically connect the first substrate 11a and the second substrate 12b together. Shapes of the plurality of ADCs 31 are the same and areas of the plurality of ADCs 31 are the same. Each ADC 31 included in the plurality of ADCs 31 is connected to a pixel block corresponding to each ADC 31 through the connection electrode 25 and the connection electrode 34. Each ADC 31 included in the plurality of ADCs 31 is connected to the connection electrode 34 at the same position in each ADC 31. The position of input terminals 36 is the same between ADCs 31 included in the plurality of ADCs 31.

In terms of points other than the above, the configuration shown in FIG. 5 is similar to the configuration shown in FIG. 4.

In the second substrate 12b, as with the second substrate 12 shown in FIG. 2, a plurality of vertical signal lines 35 may be disposed. In such a case, as with the second substrate 12 shown in FIG. 2, the connection electrode 34 may be disposed outside the plurality of ADCs 31 and the connection electrode 25 may be disposed outside the imaging unit 21. The plurality of ADCs 31 may be an AD conversion circuit using a ΔΣ method.

In the third embodiment, two pixel blocks corresponding to two ADCs 31 adjacent to each other in the column direction are adjacent to each other in the first substrate 11a. For this reason, the imaging device 10b can suppress deterioration of image quality due to the variation of property between the ADCs 31.

In a case in which the position of the input terminal 36 in each ADC 31 included in the plurality of ADCs 31 is the same, laying out the ADCs 31 is easy. However, there is a case in which the load variation occurs due to the layout of combinations of the pixel block and the ADC 31 corresponding to the pixel block.

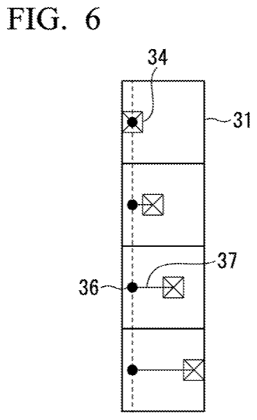

FIG. 6 shows the arrangement of the ADCs 31 in an embodiment compared to the third embodiment. In FIG. 6, the arrangement of one column in the array of the plurality of ADCs 31 is shown. As shown in FIG. 6, two ADCs 31 adjacent to each other in the column direction are not shifted from each other in the row direction. The position of the input terminal 36 in each ADC 31 is the same. The position of the connection electrode 34 is the same as the position of the connection electrode 34 shown in FIG. 5. Since the positions of connection electrodes 34 in the row direction are different from each other, a signal line 37 that connects the connection electrode 34 and the input terminal 36 together is disposed.

The signal line 37 corresponding to the ADC 31 in the first row is not disposed. The lengths of the signal lines 37 corresponding to the ADCs 31 in the second to fourth rows are different from each other. For this reason, the load of the ADC 31 is different in accordance with the row position.

In the third embodiment, two ADCs 31 adjacent to each other in the column direction are shifted from each other by a predetermined distance in the row direction in the array of the plurality of ADCs 31. For this reason, the signal line 37 is unnecessary. Even when the position of the input terminal 36 in each ADC 31 included in the plurality of ADCs 31 is the same, the imaging device 10b can suppress the load variation between the ADCs 31 due to the layout of combinations of the pixel block and the ADC 31 corresponding to the pixel block. In this way, the imaging device 10b can suppress deterioration of image quality due to the load variation between the ADCs 31.

Each ADC 31 included in the plurality of ADCs 31 is connected to the connection electrode 34 in the same position in each ADC 31. For this reason, the position of the input terminal 36 in each ADC 31 included in the plurality of ADCs 31 is the same. In this way, the imaging device 10b can suppress deterioration of image quality due to the load variation between the ADCs 31 and can make laying out the ADCs 31 easy. In addition, since the vertical signal line 35 is unnecessary, a signal line for transferring a pixel signal is short.

### Fourth Embodiment

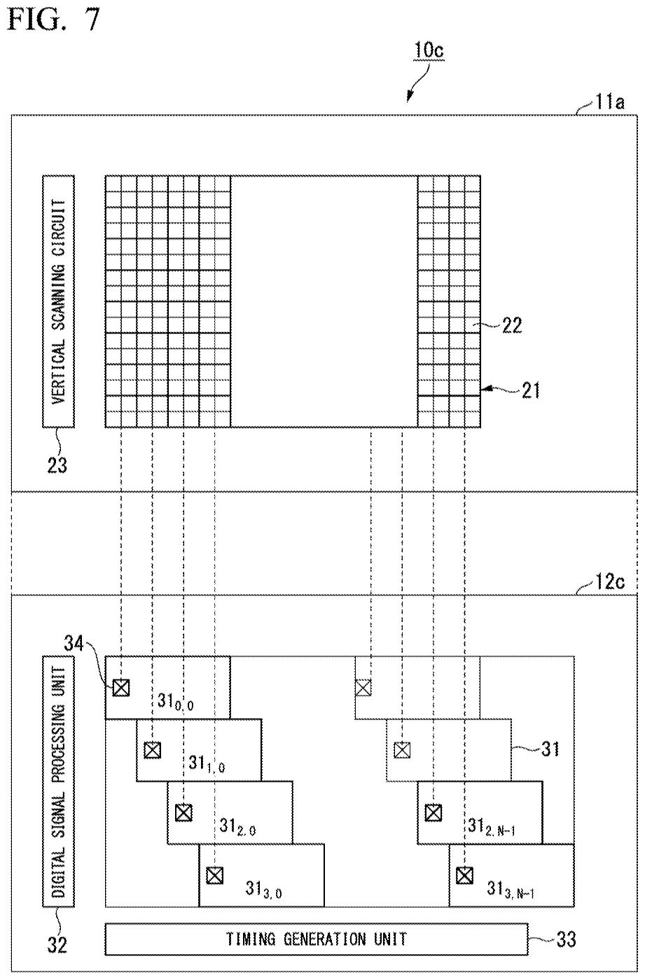

FIG. 7 shows a circuit configuration of a first substrate 11a and a second substrate 12c included in an imaging device 10c according to a fourth embodiment of the present invention. In FIG. 7, planar arrangement of circuits in the first substrate 11a and the second substrate 12c is shown. In terms of the configuration shown in FIG. 7, points different from the configuration shown in FIG. 5 will be described.

The first substrate 11a shown in FIG. 7 is the same as the first substrate 11a shown in FIG. 5. The number of pixels in the row direction is 2 m. The number m is an integer greater than or equal to 6. Each pixel block includes pixels 22 of two columns.

In the second substrate 12c, the number of columns of a plurality of ADCs 31 is m/4. The width of the ADC 31 in the row direction is eight times as large as the pitch of the pixel 22. Therefore, the width of the ADC 31 in the row direction is equal to the sum of the widths of eight pixels 22 in the row direction. For all the combinations of two ADCs 31 adjacent to each other in the column direction, two ADCs 31 adjacent to each other in the column direction are shifted from each other by a predetermined distance in a row direction in the array of the plurality of ADCs 31. The predetermined distance is twice as large as the pixel pitch.

In terms of points other than the above, the configuration shown in FIG. 7 is similar to the configuration shown in FIG. 5.

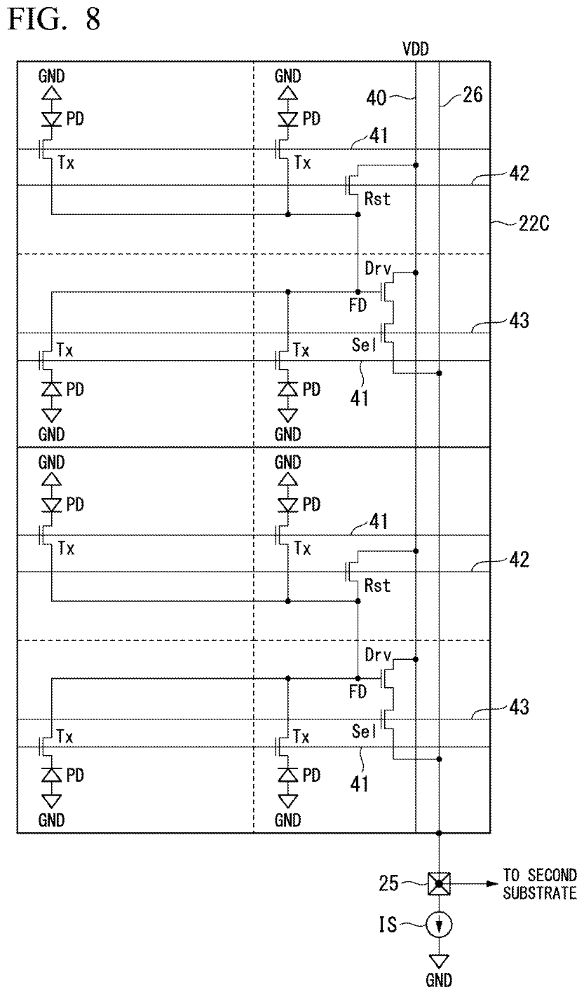

FIG. 8 shows a configuration of the pixel 22. Configurations of the eight pixels 22 are shown in FIG. 8 as a representative. Configurations of other pixels 22 are similar to the configuration shown in FIG. 8. In terms of the configuration shown in FIG. 8, points different from the configuration shown in FIG. 3 will be described.

Four pixels 22 in two rows and two columns constitute a shared pixel 22C. The shared pixel 22C shares part of the circuits included in each pixel 22. Four pixels 22 constituting the shared pixel 22C shares a floating diffusion FD, a reset transistor Rst, an amplification transistor Dry, and a selection transistor Sel.

In terms of points other than the above, the configuration shown in FIG. 8 is similar to the configuration shown in FIG. 3.

The imaging device 10c according to the fourth embodiment can suppress deterioration of image quality due to the variation of property between the ADCs 31 as with the imaging device 10b according to the third embodiment. In the fourth embodiment, an effect similar to that in the third embodiment can be obtained.

### Fifth Embodiment

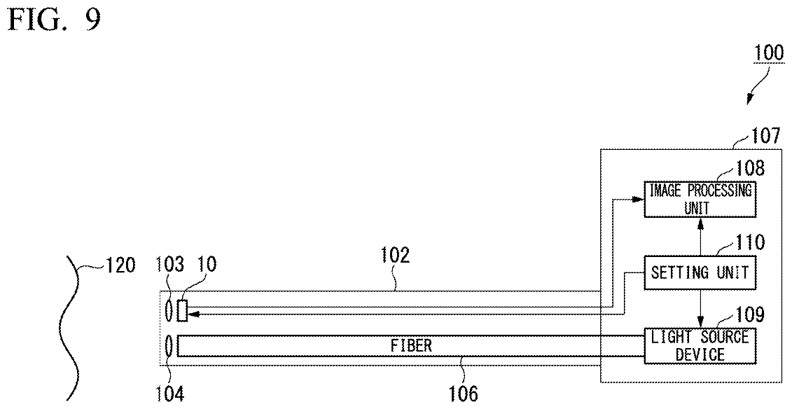

FIG. 9 shows a configuration of an endoscope device 100 according to a fifth embodiment of the present invention. The endoscope device 100 includes the imaging device 10 according to the first embodiment. As shown in FIG. 9, the endoscope device 100 includes a scope 102 and a housing 107. The scope 102 includes the imaging device 10, a lens 103, a lens 104, and a fiber 106. The housing 107 includes an image processing unit 108, a light source device 109, and a setting unit 110.

The lens 103 forms an image of reflected light from a subject 120 on the imaging device 10. The fiber 106 transfers illumination light with which the subject 120 is irradiated. The lens 104 irradiates the subject 120 with the illumination light transferred by the fiber 106. The light source device 109 includes a light source that generates the illumination light with which the subject 120 is irradiated. The image processing unit 108 generates a captured image by performing predetermined processing on a signal output from the imaging device 10. The setting unit 110 controls an imaging mode of the endoscope device 100.

The configuration of the endoscope device 100 is not limited to the above-described configuration. The endoscope system according to each aspect of the present invention may not include a configuration corresponding to at least one of the lens 103, the lens 104, the fiber 106, the image processing unit 108, the light source device 109, and the setting unit 110.

Instead of the imaging device 10, any one of the imaging device 10a shown in FIG. 4, the imaging device 10b shown in FIG. 5, and the imaging device 10c shown in FIG. 7 may be used.

The endoscope device 100 according to the fifth embodiment includes the imaging device 10 for which deterioration of image quality is suppressed. For this reason, the endoscope device 100 can suppress deterioration of image quality.

While preferred embodiments of the invention have been described and shown above, it should be understood that these are examples of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. An imaging device comprising:
a first substrate; and
a second substrate stacked on the first substrate,
wherein the first substrate includes a plurality of pixels forming a first array and disposed in a matrix shape,
each pixel included in the plurality of pixels belongs to one pixel block included in a plurality of pixel blocks and is configured to output an analog pixel signal,
the second substrate includes a plurality of AD conversion circuits forming a second array, each AD conversion circuit included in the plurality of AD conversion circuits being configured to convert the pixel signal read from two or more pixels included in the plurality of pixels and belonging to the pixel block corresponding to the AD conversion circuit to a digital signal,
at least one of the first substrate and the second substrate includes a selecting circuit configured to control a timing at which the pixel signal is read from the plurality of pixels,
each pixel block included in the plurality of pixel blocks includes all of the pixels disposed in one or more columns in the first array,
the plurality of AD conversion circuits are disposed in a matrix shape having M rows and N columns, the M being an integer greater than or equal to 3, the N being an integer greater than or equal to 2,
a width of each AD conversion circuit included in the plurality of AD conversion circuits in a row direction in the second array is larger than a pitch of each pixel included in the plurality of pixels, and
for all combinations of two pixel blocks included in the plurality of pixel blocks and adjacent to each other in the first substrate, two AD conversion circuits included in the plurality of AD conversion circuits and corresponding to the adjacent two pixel blocks are adjacent to each other in the second substrate.

2. The imaging device according to claim 1,
wherein the plurality of pixel blocks include a first pixel block, a second pixel block, a third pixel block, and a fourth pixel block,
the second pixel block is adjacent to the first pixel block in a row direction in the first array,
the fourth pixel block is adjacent to the third pixel block in the row direction in the first array,
the plurality of AD conversion circuits include a first AD conversion circuit corresponding to the first pixel block, a second AD conversion circuit corresponding to the second pixel block, a third AD conversion circuit corresponding to the third pixel block, and a fourth AD conversion circuit corresponding to the fourth pixel block,
the second AD conversion circuit is adjacent to the first AD conversion circuit in a column direction in the second array,
the fourth AD conversion circuit is adjacent to the third AD conversion circuit in a direction opposite to the column direction in the second array, and
a first column including the first AD conversion circuit and the second AD conversion circuit is adjacent to a second column including the third AD conversion circuit and the fourth AD conversion circuit in the second array.

3. The imaging device according to claim 1, further comprising a connection electrode electrically connecting the first substrate and the second substrate together,
wherein the pixel that belongs to each pixel block included in the plurality of pixel blocks is connected to a signal line disposed in the first substrate,
the connection electrode is disposed so as to overlap the AD conversion circuit included in the plurality of AD conversion circuits and is connected to the signal line, and
each AD conversion circuit included in the plurality of AD conversion circuits is connected to the connection electrode.

4. An imaging device comprising:
a first substrate; and
a second substrate stacked on the first substrate,
wherein the first substrate includes a plurality of pixels forming a first array and disposed in a matrix shape,
each pixel included in the plurality of pixels belongs to one pixel block included in a plurality of pixel blocks and is configured to output an analog pixel signal,
the second substrate includes a plurality of AD conversion circuits forming a second array, each AD conversion circuit included in the plurality of AD conversion circuits being configured to convert the pixel signal read from two or more pixels included in the plurality of pixels and belonging to the pixel block corresponding to the AD conversion circuit to a digital signal,
at least one of the first substrate and the second substrate includes a selecting circuit configured to control a timing at which the pixel signal is read from the plurality of pixels,
each pixel block included in the plurality of pixel blocks includes all of the pixels disposed in one or more columns in the first array,
the plurality of AD conversion circuits are disposed in a matrix shape having M rows and N columns, the M being an integer greater than or equal to 3, the N being an integer greater than or equal to 2,
a width of each AD conversion circuit included in the plurality of AD conversion circuits in a row direction in the second array is larger than a pitch of each pixel included in the plurality of pixels,
for all combinations of two AD conversion circuits included in the plurality of AD conversion circuits and adjacent to each other in a column direction in the second array, two pixel blocks included in the plurality of pixel blocks and corresponding to the two AD conversion circuits adjacent to each other in the column direction are adjacent to each other in the first substrate, and
for all the combinations, the two AD conversion circuits adjacent to each other in the column direction are shifted from each other by a predetermined distance in a row direction in the second array.

5. The imaging device according to claim 4,
wherein the predetermined distance is integer times as large as the pitch.

6. The imaging device according to claim 4, further comprising a connection electrode electrically connecting the first substrate and the second substrate together,
wherein shapes of any two AD conversion circuits included in the plurality of AD conversion circuits are the same,
areas of any two AD conversion circuits included in the plurality of AD conversion circuits are the same,
each AD conversion circuit included in the plurality of AD conversion circuits is connected to the pixel block corresponding to the AD conversion circuit through the connection electrode, and
each AD conversion circuit included in the plurality of AD conversion circuits is connected to the connection electrode at the same position in the AD conversion circuit.

7. The imaging device according to claim 5,
wherein each AD conversion circuit included in the plurality of AD conversion circuits is configured to be an AD conversion circuit using a ΔΣ method.

8. An endoscope device comprising an imaging device according to claim 1.
